(19) United States
(12) Patent Application Publication
(10) Pub. No.: US 2023/0032996 A1
(43) Pub. Date: Feb. 2, 2023

(54) System and Method for Height Determination

(71) Applicant: United Metal Fabricators, Inc. d/b/a UMF Medical, Johnstown, PA (US)

(72) Inventors: Joseph Romano, Johnstown, PA (US); Quinn Carpenter, Johnstown, PA (US)

(73) Assignee: United Metal Fabricators, Inc. d/b/a UMF Medical, Johnstown, PA (US)

(21) Appl. No.: 17/383,984

(22) Filed: Jul. 23, 2021

(51) Int. Cl. A61B 5/107; G01B 11/02; G01S 17/26; G01S 7/48; G01S 7/497

(52) U.S. Cl. CPC A61B 5/1072; G01B 11/02; G01S 17/26; G01S 7/4808; G01S 7/497

(57) ABSTRACT

Systems and methods for determining the height of a subject include a fixed unit mounted to a surface in electronic communication with a mobile unit permitting user input to drive instructions for the system. A sensor in the fixed unit emits a signal upon activation which is received. Time difference between emitting and receiving the signal is used to calculate the height of the room for calibrating the system and the height of the subject when measuring. The mobile unit includes a measurement surface positioned proximate to a maximal point of the subject for measurement, which intercepts and preferably reflects a measurement signal emitted by the sensor of the fixed unit. A light source in the fixed unit may create a mark on the support substrate indicating the location for the subject for measurement. The height determination may be sent to a hub or subject's EMR.

### FIELD OF THE INVENTION

This invention relates to height determination systems, and more particularly, to height determination systems involving communication between devices.

### BACKGROUND

Conventional height determination systems long used in doctor's offices and other medical settings include manual measuring of a subject's height against a vertical rail or ruler with a horizontal arm pressed against the top of the person's head. However, these manual measurements can be inaccurate. It is very easy for the arm placed at the top of the subject's head to be positioned at an angle rather than truly horizontal, such as because of height differences between the user taking the measurement and the subject being measured. This can occur even when the measurement arm is movably affixed along a track superimposed on the vertical ruler.

Recent developments have moved toward digital height measurement systems to avoid this user-generated error from manual measurements. Some use sensors that measure the subject's height to the floor, as the manual versions do. However, these sensors cannot be placed directly on the subject's head since they require a clear line of sight to the floor for measurement. Other objects such as bags and nearby furniture may also interfere with such downward-facing digital measurement systems.

Others have gone in the opposite direction and measure between the subject's head and the ceiling of the room, as in U.S. Pat. No. 9,026,392. In this patent, a single device is first placed on the floor of the room and a laser beam is directed to the ceiling to determine the height of the room for calibration. The same device is then placed on top of the patient's head and the laser beam is again directed to the ceiling. The difference in the second measurement from the calibration measurement provides the patient's height. However, the '392 patent also discloses that the angle of measurement is often not exactly vertical, being slightly angled when placed on the subject's head. In such cases, an inclinometer is used to determine the angle of deviation and trigonometry is then used to determine the straight vertical height.

Still others have used downward-facing sensors to automatically measure the height of a person, as in U.S. Pat. No. 5,763,837. In this patent, a human subject stands on a scale with an integrated weight sensor to obtain a weight measurement. Simultaneously, a sonar head positioned in a stationary position above the weight scale is activated to emit sound waves from a sound wave emitter, which are received by a plurality of sound wave receptors. The sound waves bounce off the subject's head and return to the sonar head for detection. The measurement process is repeated four times. Software is used to average the various measurement runs and calculate the weight and height of the subject. The downside of this height measurement system is that the sound waves reflect off the highest point having mass on the person's head. This means that the height measured is to the top of the person's hair, not necessarily their skull. Voluminous or vaulted hair styles may render inaccurate height determinations as a result.

Therefore, there remains improvement in the field to easily and accurately measure the height of a subject that did not involve errors or correcting for same.

### SUMMARY

The present invention is directed to systems and methods for height determination of a subject. The system includes a fixed unit mounted to a surface of a room, such as a ceiling, and a mobile unit selectively movable in the room relative to the subject and through which the system is preferably controlled. The mobile unit and fixed unit are in electronic communication with one another, either wired or wirelessly, to transmit information and signals between them. The height of a subject is calculated from measurements between the fixed and mobile units.

Each of the fixed and mobile units include a transceiver through which such information and signals are transmitted. Each device also includes a logic board with a processor and memory which operates and controls the various components and functions of the device. Each device may also be in communication with a hub for central processing and computation and/or the subject's electronic medical record (EMR) which may be hosted remotely such as in the cloud in compliance with HIPAA or other security regulations for medical data.

The mobile unit is positioned in proximity to the highest or maximal point of the subject for height determination, such as the top of the subject's head or adjacent thereto. Specifically, the mobile unit includes a measurement surface that is placed on or in proximity to the subject's head for measurement. In at least one embodiment, this measurement surface extends from the body of the mobile unit, such as an arm or paddle, and may be selectively attachable to and releasable from the mobile unit. In other embodiments, the measurement surface may be an integral component of the mobile unit, such as but not limited to a screen or portion of the body or housing thereof.

The mobile unit also includes an input sensor that receives input from a user of the device. When activated, the input sensor provides corresponding instructions to the logic board of which mode of the system is being activated—calibration, marking or measurement. In at least one embodiment there are multiple input sensors, such as each corresponding to a different mode of activation. In other embodiments, a single input sensor may be activated differently, such as by holding for a certain length of time or simply pressing, to differentiate between modes of activation. There may also be a combination of some dedicated input sensors and others that are activated differently for different modes of operation. The input sensors are accessible at the exterior of the mobile unit, such as buttons or areas on a screen responsive to haptic feedback. The mobile unit may also include a display presenting information to the user, such as but not limited to the current mode of activation, input sensors, or the resulting height as determined by the system following use.

The fixed unit may include a light source such as but not limited to a laser, light emitting diode (LED) or other source of light. The light source generates and emits a light when activated by the fixed unit logic board. This light is directed at the support substrate of the room directly or substantially directly underneath the light source, producing a mark on the support substrate from the light ray. This mark indicates where the subject is to stand or be positioned for height determination. In at least one embodiment, this mark is temporary, lasting long enough to provide an indication for positioning but then ceasing for the rest of the height determination process. It may also persist for the duration of the height determination process.

The fixed unit also includes a sensor that is configured to both transmit and receive signals of a predetermined frequency, such as but not limited to in the ultrasonic and visible light ranges. When activated by a signal from the logic board, the sensor generates and emits a signal, which may be a calibration signal or a measurement signal depending on the instructions from the logic board. The signal is emitted from the sensor outwardly toward the room, and preferably directly toward the support substrate. When calibrating the system, the calibration signal is reflected by the surface of the support substrate of the room underneath the fixed unit and the return calibration signal is received by the same sensor. When using the system to make a height determination, the measurement signal that is emitted may be reflected back to the sensor by a measurement surface of the mobile unit positioned on the subject's head. This reflection of the measurement signal may be active or passive. Alternatively, the measurement surface may simply intercept and register the measurement wave without reflecting it back to the sensor. At least one of the sensor and/or logic board tracks the time between emitting and receiving the signal. This time difference is used to determine the height of the room to calibrate the system, and the distance to the subject when measuring for height determination.

The present invention is also directed to methods of determining the height of a subject using the system described herein. The method includes calibrating the system to determine the room height, marking a surface to indicate positioning of the subject, and measuring the height of the subject. In each step, the fixed unit emits a corresponding signal upon receipt of an initiation signal, which may be based on user input. Calibration of the system includes transmitting an initiation signal from the mobile unit to the fixed unit, which is then decoded as a calibration initiation signal. Operative instructions are sent to the sensor to emit a calibration signal. The calibration signal is directed at the support substrate of the room where the fixed unit is mounted. The calibration signal bounces off the support substrate and is returned to the sensor. The time it takes the calibration signal to traverse the length of the room and return is recorded and used to calculate the height of the room, which is saved as a known preselected distance for future use. In some embodiments, calibration may not be needed such as when the height of the room, i.e., the distance between the fixed unit and support substrate, is known and is stored in memory.

Marking includes receiving input from a user and transmitting a corresponding initiation signal from the mobile unit to the fixed unit where it is decoded. Operative instructions are sent to the light source of the fixed unit when the initiation signal is decoded as a marking initiation signal to activate the light source for a predetermined length of time, emitting a visible light directed at the support substrate underneath the fixed unit. When the light hits the support substrate, it creates a mark on the support substrate indicating where the subject should stand. In at least one embodiment it is temporary, lasting a few seconds. In some embodiments, the support substrate may be a hub such as a medical examination table.

Measuring includes positioning the mobile unit relative to the subject, such as on top of, proximate or adjacent to the top of the subject's head. Input is received from the user, generating and transmitting an initiation signal from the mobile unit to the fixed unit where it is decoded. Operative instructions are sent to the sensor of the fixed unit to emit a measurement signal when the initiation signal is a measurement initiation signal. The measurement signal is intercepted by the measurement surface of the mobile unit. In at least one embodiment, the measurement signal is reflected back to the sensor by the measurement surface of the mobile unit. In other embodiments, the measurement signal may be received and detected by the mobile unit, such as by the measurement surface. The time between emitting and receiving the measurement signal is used to determine a distance to the maximal point of the subject, which is subtracted from the known preselected distance or height of the room to determine the height of the subject. Either the mobile unit, fixed unit, or hub may perform these calculations. By using a surface-mounted source for the measurement signal, errors from angular deviation—and having to compensate for the same—are avoided.

The height determination systems and methods, together with their particular features and advantages, will become more apparent from the following detailed description and with reference to the appended drawings.

### DESCRIPTION OF THE DRAWINGS

FIG. 1A is a schematic diagram of a system for determining the height of a subject of the present invention.

FIG. 1B is a schematic diagram of the system of the present invention using a second embodiment of measurement surface on the mobile unit.

FIG. 2 is a schematic diagram of the components of the fixed and mobile units of the system.

FIG. 3 is a schematic diagram of the system when calibrating.

FIG. 4 is a schematic diagram of the system when marking the location for subject positioning.

FIG. 5 is a schematic diagram of the system when measuring and determining the height of the subject.

FIG. 6 is a schematic diagram of a method of determining the height of a subject of the present invention.

FIG. 7 is a schematic diagram of the steps of calibrating the system.

FIG. 8 is a schematic diagram of the steps of marking, measuring and determining the height of the subject.

Like reference numerals refer to like parts throughout the several views of the drawings.

### DETAILED DESCRIPTION

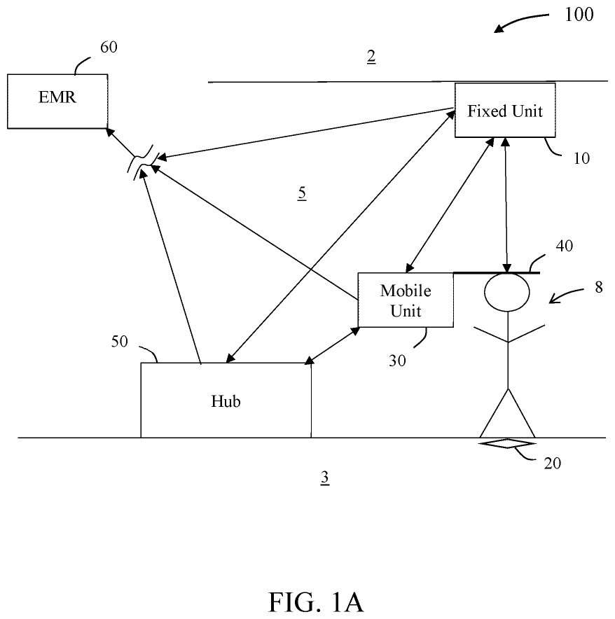

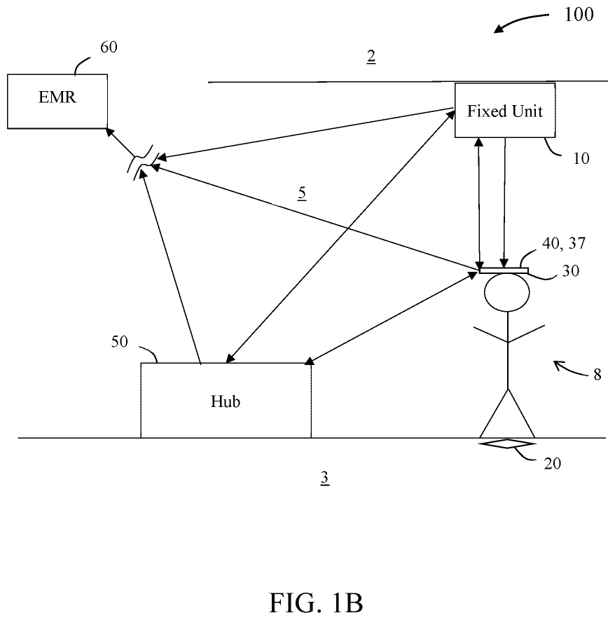

As shown in the accompanying drawings, the present invention is directed to a system and method for wirelessly determining the height of a subject. Specifically, the system 100 is deployable within a space 5 having a surface 2 and a support substrate 3, as shown in FIG. 1A. In at least one embodiment, the space 5 may be a room, the surface 2 may be a ceiling and the support substrate 3 may be a floor, though these should not be limiting. For instance, the space 5 may be a medical examination room or a triage area within a medical practice or hospital, though in other embodiments the space 5 may be a non-medical room such as within a residential, business, school, military, warehouse or non-profit facility. In still other embodiments, the space 5 need not be enclosed, such as in a field-operated or emergency structure. Similarly, the surface 2 may be any surface of the space 5, which may include a wall or other structural boundary. The support substrate 3 may be any surface on which the subject may be supported, including but not limited to the floor, ground or equipment located within the space 5, such as a hub 50 as discussed below which may be a medical examination table or other equipment, which need not be medical in nature. These are a few non-limiting examples.

The system 100 includes a fixed unit 10 mounted to the surface 2 of the space 5. The fixed unit 10 faces toward the interior of the space. As will be discussed in greater detail below, the fixed unit 10 emits and receives signals transmitting information. These signals may include, but are not limited to, waves in the ultrasonic, sonic, visible light, UV light, and other light ranges, and may be amplified and/or collimated such as through a laser. In at least one embodiment, the fixed unit 10 emits ultrasonic signals through a laser. These signals are emitted from the fixed unit 10 directed inwardly toward the space 5. When a subject 8 whose height is desired to be known is present within the space 5, the signals are emitted from the fixed unit 10 inward to the room toward a predetermined location at which the subject is positioned. A mark 20 may be displayed on the support substrate 3 by the fixed unit 10 showing a user 6 where to direct the subject 8 to stand. When the subject 8 is positioned on, over or within the location of the designated mark 20, the signals emitted from the fixed unit 10 are directed toward the subject 8. When the fixed unit 10 is mounted to a ceiling, the signals emitted therefrom are directed toward the subject's head. The subject 8 may be a human, animal or even object of whose height is desired to be known or determined and may be any age. Though described herein as a height, the same system can be used to measure any dimension of the subject 8. For instance, when the fixed unit 10 is mounted to a surface 2 that is a wall or other side boundary of the space 5, the signals it emits may be used to determine a length, width or depth of the subject 8 depending on how the subject 8 is oriented within the space 5 and relative to the fixed unit 10.

The system 100 also includes a mobile unit 30 in electronic communication with the fixed unit 10 and exchanges information therewith. This electronic communication may be wireless, such as by Wi-Fi, BlueTooth®, RF and RFID, or may be wired. The mobile unit 30 may be an electronic device that may be utilized by a user 6 when triaging the subject 8, obtaining the vital signs and basic physiological information of the subject 8, or before, during or after examining the subject 8. For instance, in at least one embodiment the mobile unit 30 may be an electronic tablet, electronic pad, smartphone, or other interactive handheld electronic device. The mobile unit 30 may be placed in proximity to the subject 8 when using the height determination system 100, such as on top of and/or beside a maximal point of the subject 8 such as the subject's head. It should be appreciated that FIG. 1A is not drawn to scale but is provided with the fixed unit 10 and mobile unit 30 exaggerated for the purposes of demonstration and explanation.

In at least one embodiment, the mobile unit 30 may include a measurement surface 40 which extends on or from the mobile unit 30 and may be placed on top of the subject's head during use of the height determination system 100, as shown in FIG. 1A. The measurement surface 40 may extend laterally, longitudinally or radially relative to the mobile unit 30. In at least one embodiment, the measurement surface 40 has an elongate shape, such as but not limited to a rectangle, square, oval, or irregular shape. The length of the measurement surface 40 may be at least as long as the width of the subject's head in at least one embodiment. For instance, in at least one embodiment, the measurement surface 40 may be a paddle, stick, arm, or other member having a length which extends from the mobile unit 40 and may extend in a cantilevered fashion. The measurement surface 40 may be affixed to the mobile unit 30, such as to a housing thereof, either permanently or temporarily through removable attachment such as but not limited to hook-and-loop, snap-fit, frictional fit or magnetic fasteners. Temporary securing to the mobile unit allows the measurement surface 40 to be removed from the mobile unit 30 when its use is not desired, such as when the mobile unit 30 is not being used for height determination. The measurement surface 40 may be made of a material capable of reflecting the waves emitted from the fixed unit 10 back to the fixed unit 10, either actively or passively. Examples include, but are not limited to, glass, plastic, polymers, wood, metal and may be opaque, translucent or transparent. In at least one embodiment, the measurement surface 40 may be plastic or thermoplastic such as but not limited to acrylonitrile butadiene styrene (ABS) and polypropylene, which may be formed by methods such as but not limited to injection molding, thermoforming, additive or deposition techniques. In still other embodiments, the measurement surface 40 may be metal or combination of metals and/or metal alloys. In some embodiments, the measurement surface 40 may include a detector 37 which receives the measurement signal and registers it rather than reflecting it back to the fixed unit 10, such as in FIG. 1B.

In some embodiments, such as shown in FIG. 1B (also not drawn to scale), the measurement surface 40 may be a component part of the mobile unit 30 itself, such as the housing, screen or touchscreen thereof. Accordingly, in such embodiments, the measurement surface 40 may be integrally formed with the mobile unit 30 and does not extend therefrom. In such embodiments, the mobile unit 30 itself may be placed on top of the subject's head during for height measurement and determination using the present system 100. For instance, the mobile unit 30 may be positioned flat against the top of the head of the subject 8 with the housing or screen of the mobile unit 30 facing the fixed unit 10 mounted above. The housing or screen of the mobile unit 30 then intercepts the measurement signal, either receiving it from or reflecting it back to the fixed unit 10.

The mobile unit 30 is preferably used to control the system 100, with a user 6 providing input to the mobile unit 30 to direct the emission of waves from the fixed unit 10. Accordingly, the mobile unit 30 is in electronic communication with the fixed unit 10, capable of sending and receiving information to and from the fixed unit 10. However, in some embodiments, certain functions of the fixed unit 10 may occur automatically, such as calibrating the system 100 when a preset period of time has elapsed since the last calibration or emitting a measurement signal automatically once a presence is detected at the predetermined location which may be regularly monitored by the fixed unit 10. These are but a few illustrative and non-limiting examples.

In some embodiments, the system 100 also includes a hub 50 in electronic communication with at least one of, if not both, the fixed unit 10 and mobile unit 30. The hub 50 may be located in the same space 5 as the fixed unit 10 or in a different room or space that is within electronic communicating distance of the fixed unit 10 and/or mobile unit 30. The hub 50 may itself be an electronic device coordinating both the mobile unit 30 and fixed unit 10, or at least receiving information from the mobile unit 30 and/or the fixed unit 10. For instance, the hub 50 may be a computer or workstation used by nurses or other medical professionals in assisting with or preparing for examination of the subject. In certain embodiments, the hub 50 may be an examination table, procedure chair, or other furniture that is equipped with computing hardware and software, such as a processor, memory and wireless communication such as but not limited to Wi-Fi, BlueTooth®, RF and RFID, allowing the furniture to itself be a “smart” device. In some embodiments, the hub 50 or a portion thereof may be the predetermined location on which the subject 8 is positioned for height determination. Regardless of form, the hub 50 may in turn be in electronic communication to a network, such as but not limited to a local area network (LAN), wide area network (WAN), Wi-Fi network, enterprise network or other network allowing access to other computing devices nearby and/or the Internet. The hub 50 may also have processing capabilities to determine the body mass index (BMI) value of the subject 8 upon receipt of the height as determined by the system 100, as explained in greater detail below, in conjunction with a weight measurement determined separately, such as from a scale in electronic or digital communication with the hub 50. In some embodiments, the hub 50 itself may be able to make the weight determination, such as when the hub 50 is an examination table having an integrated scale that weighs the subject 8 when they sit or lie on the table. This may occur separately from or in conjunction with the height determination measurement. These are but a few embodiments provided for illustrative purposes.

At least one or each of the fixed unit 10, mobile unit 30 and hub 50 may be in electronic communication with a network and/or the Internet, as described above, in order to convey the height measurement (and/or BMI value derived therefrom) to the electronic medical record (EMR) 60 of the subject 8, as shown in FIG. 1A. The EMR 60, which may also be referred to interchangeably as an electronic health record (EHR), may be hosted elsewhere, such as may be cloud hosted or stored on servers for the medical practice or another medical practice. As such, the height measurement as determined by the system 100 may be provided to the EMR 60 through an Internet connection for updating the record. Thus, the subject's treating physicians may have access to this information, even if they were not the ones that captured the information using the system 100.

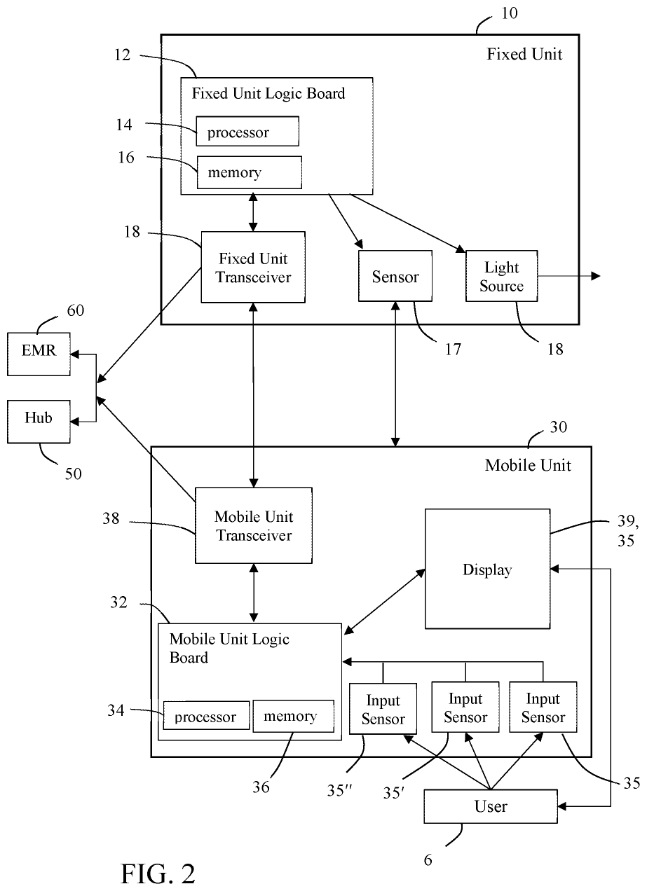

Now that the overall system 100 has been described generally, the specific components will be discussed in greater detail. As shown in FIG. 2, the fixed unit 10 includes an fixed unit logic board 12 that coordinates signals and information flow between the various other hardware in the fixed unit 10. For instance, the fixed unit 10 includes a processor 14 that performs executable instructions on data and electronic signals. The processor 14 may be any suitable processor having sufficient power and processing capabilities to operate the components of the fixed unit 10 and/or perform the calculations described herein. Examples include but are not limited to a microprocessor, microcontroller, ARDUINO®, embedded processor, and digital signal processor (DSP), and may be a chip(s) such as but not limited to wireless chip(s), board(s) such as but not limited to breadboard(s) and printed circuit board(s) (PCBs). In at least one embodiment, the processor 14 is a microprocessor such as the ATmega328 (Microchip Technology Inc., of Chandler, Ariz.) RISC-based microprocessor with a 16-20 MHz clock speed, CPU speed of 20 MIPS/DMIPS, at least 8-bit BUS width and operating at 1.8-5.5 volts, though other processor types are also contemplated. The processor 14 may have programmable elements for selective configuration, which may be configured at the manufacturer of the fixed unit 10 or in the field upon installation or use, such as but not limited to through programmable ports. For instance, in at least one embodiment, the processor 14 may be an ARDUINO® programmable to send and receive signals to and from the mobile unit 30, other components of the fixed unit 10, the hub 50 and EMR 60. A non-limiting illustrative example includes an Arduino Nano (Arduino AG, Switzerland) operating with 5V, 32 Kb flash, 2 Kb SRAM, 8 Input Pins, 22 Digital Out Pins and 6 PWM pins.

The fixed unit 10 may also include memory 16 that stores executable instructions, programs, information and other data. The memory 16 may be onboard memory that is part of the processor 14, or it may be a separate component that is electrically accessed by the fixed unit logic board 12. The memory 16 may be primary storage or temporary storage memory. The memory 16 may be a chip(s) installed on the fixed unit logic board 12 and accessed by the processor 14 during use of the fixed unit 10. The memory 16 may be random access memory (RAM) such as but not limited to dynamic RAM (DRAM), synchronous dynamic RAM (SDRAM), static RAM (SRAM), ferroelectric RAM (FRAM) and magneto-resistive RAM (MRAM), having a double data rate of DDR1, DDR2, DDR3, DDR4 or DDR5; read only memory (ROM) such as but not limited to programmable read-only memory (PROM), erasable programmable read only memory (EPROM) and electrically erasable programmable read only memory (EEPROM); complementary metal-oxide-semiconductor (CMOS) and flash. The memory 16 has at least enough storage capacity to save one floating value, such as but not limited to 16 bytes of data, which may be long term or short-term storage. In at least one embodiment, the memory 16 includes 1 KB EEPROM and integrated into a breadboard or PCB using ARDUINO® logic. As noted above, the memory 16 stores executable instructions, programs, information and other data accessed and used by the fixed unit logic board 12 during use. For example, the executable instructions for sending and receiving signals and operative instructions to the other components of the fixed unit logic board 12 may be included in memory 16. The algorithm(s) and/or rules used to interpret incoming data, make calculations and determine height from the measurements received, which are discussed in greater detail below, may also be stored in the memory 16 and accessed by the processor 12 for use. The calibration measurements and information, such as room height, may be stored in the memory 16 and may be rewritten on demand with subsequent calibration steps. In some embodiments, the calibration measurements and information may be transmitted to the mobile unit 30 for storage and calculation performance, as described in detail below. These are but a few non-limiting examples.

The fixed unit 10 also includes a sensor 17 in electronic communication with the fixed unit logic board 12. The sensor 17 sends and receives measurement and/or calibration signals from the fixed unit 10. For instance, in at least one embodiment the sensor 17 is a wave emitter such as but not limited to an HC-SR04 ultrasonic range module (ELECFREAKS, Shenzhen, China) providing 40 kHz pulses over a range of 2 cm to 4 meters when activated, though other ultrasonic modules with differing operative parameters are also contemplated. Additional examples include but are not limited to the LIDAR Lite v3 (GARMIN®, Olathe, Kans.) emitting laser light at 905 nm (1.3 watts) with 4 m Radian×2 m Radian beam divergence and an optical aperture of 12.5 mm, though other LIDAR models are also contemplated herein. As noted above, the signal emitted by the fixed unit 10 may be a wave in the ultrasonic, sonic, visible light, infrared (IR), ultraviolet (UV), and other light ranges, and may be amplified and/or collimated such as through a laser. For example, the sensor 17 may be a laser or light emitting diode (LED), as a few non-limiting examples. The sensor 17 may emit the signal in a constant wave for a predetermined length of time, such as up to 100 microseconds, or as a pulsed wave with each pulse having a duration in the range of up to 10 microseconds with a pause of up to 10 microseconds between pulses. In at least one embodiment, the sensor 17 emits the signal as a constant wave at a frequency of about 40 kHz for a predetermined length of time, such as 10 microseconds. In addition to emitting the signal, the sensor 17 also detects signals of a predetermined frequency or frequency range, preferably of the same frequency or range as those it emits, though it may detect signals of different frequencies than those emitted. In at least one embodiment, the signal detected by the sensor 17 is the same signal(s) that was emitted from the sensor 17 reflected back by an object, such as the mobile unit 30, measurement surface 40 thereof and/or support substrate 3.

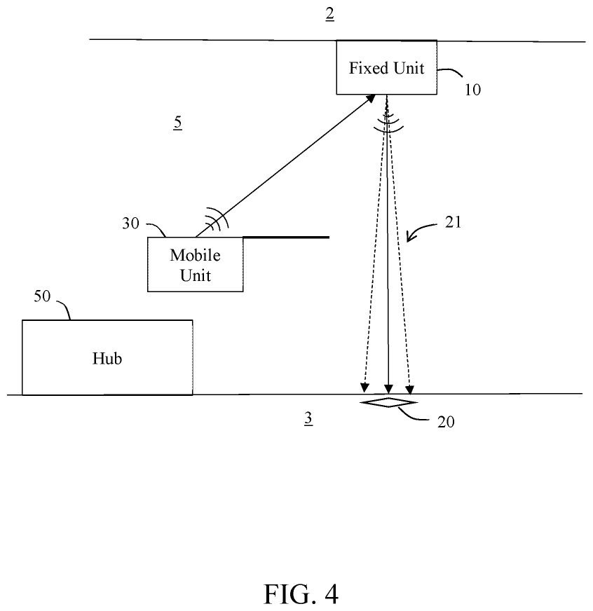

The fixed unit 10 also includes a light source 18 that emits light of a predetermined wavelength when activated. The light source 18 is positioned within the fixed unit 10 so it emits light directed inwardly to the space, and specifically to the support substrate 3 of the space 5, where it appears as a mark 20 on the support substrate 3, as shown in FIG. 1A. This mark 20 indicates where the subject 8 should stand for measuring and determining their height using the present system 100. In at least one embodiment, light is emitted from the light source 18 substantially perpendicular to the support substrate 3 underneath the fixed unit 10, such as may be directly underneath the fixed unit 10 when the support substrate 3 is a floor as shown in FIG. 4. In some embodiments, light may be emitted from the light source 18 in a cone substantially perpendicular to the support substrate 3 underneath the fixed unit 10 covering a larger diameter on the support substrate 3 than is emitted from the light source 18. In such embodiments, the cone of emitted light may be at angles of up to about 45° deviating in any direction from perpendicular to the light source 18 and/or support substrate 3. The light source 18 may be any light source that is selectively activatable and emits light when activated, such as but not limited to light emitting diode(s) (LED), laser or laser diode, incandescent or filament bulbs, fluorescent bulbs and compact fluorescent light bulbs (CFL). The light source 18 may be capable of producing and emitting a light, such as but not limited to a 5 mW laser diode operating on 5V, though more powerful diodes and light sources are also contemplated. The light emitted from the light source 18 may be of a predetermined frequency or range of frequencies, such as may be determined by the bulb or diode used. Preferably, the light emitted from the light source 18 is in the visible light spectrum, such as having a wavelength in the range of 380 to 700 nanometers. In other embodiments, the light emitted from the light source 18 may be in the infrared (IR), ultraviolet (UV) or other light spectra. The light produced may be any color and the light source 18 may include a lens or filter to create a particular pattern with the light as it is emitted. The light emitted from the light source 18 may be the same or different type, frequency or wavelength of wave emitted from the sensor 17. In a preferred embodiment, the light source 18 produces a visible light ray whereas the sensor 17 produces an ultrasonic wave.

As shown in FIG. 2, the fixed unit 10 also includes a fixed unit transceiver 19 providing electronic and/or digital communication with other devices and components of the system 100, including the mobile unit 30, hub 50 and EMR 60. The fixed unit transceiver 19 may include hardware and software enabling transmission and receipt of electronic and/or signals and packets of information. Such communication may occur over a network, such as a closed network as a LAN (which may be wired or wireless) or Wi-Fi network, or it may occur through near-field technology such as but not limited to Bluetooth®, radio frequency (RF) and radio-frequency identification (RFID). In at least one embodiment, the fixed unit transceiver 19 may be a wireless communication module enabling wireless communication with other enabled devices for the exchange of information between the devices. Examples include but are not limited to a wireless chip, Wi-Fi chip or card, Bluetooth® chip and RFID tag. In some embodiments, the hardware for the fixed unit transceiver 19, such as chips or tags, may be integrated into the fixed unit logic board 12 or processor 14, and the software for the fixed unit transceiver 19 may be stored in the memory 16 discussed above. In other embodiments, the hardware and software comprising the fixed unit transceiver 19 may be physically separate from the fixed unit logic board 12, processor 14 and memory 16 discussed above, such as when the fixed unit transceiver 19 is a standalone component with its own integrated on-board memory and/or processor. In such embodiments, the fixed unit transceiver 19 is in electrical communication with the fixed unit logic board 12, sending and receiving signals and information with the fixed unit logic board 12. Regardless of location and/or integration, in at least one embodiment, the fixed unit logic board 12 controls and directs the operation of the fixed unit transceiver 19.

As shown in FIG. 2, the mobile unit 30 of the height determination system 100 includes some components that are similar to the fixed unit 10. For instance, as shown in FIG. 2, the mobile unit 30 includes a mobile unit logic board 32 having a processor 34 and memory 36, and a mobile unit transceiver 38. These may be as described above for the fixed unit logic board 12, processor 14, memory 16 and fixed unit transceiver 19, and may be the same or different as the particular fixed unit logic board 12, processor 14, memory 16 and fixed unit transceiver 19 selected for use in the fixed unit 10. For instance, the mobile unit logic board 32 includes logic and circuitry for directing the operation of the mobile unit 30 and for directing communications to and interpreting information from the fixed unit 10, hub 50 and EMR 60 through the mobile unit transceiver 38. In at least one embodiment, the mobile unit logic board 38 includes ARDUINO® logic operating through circuitry on a breadboard or PCB as a processor 34, the memory 36 is EEPROM memory installed on the breadboard or PCB, and the mobile unit transceiver 38 is a wireless chip installed thereon. In some embodiments, the calculations for determining height, as described below, may be performed by the processor 34 on the mobile unit 30. For instance, the fixed unit 10 may transmit at least the data including the time of emitting and detecting the measurement signal for height measurement determination, or the difference in time thereof, and may also transmit the baseline or calibration data for the distance between the fixed and mobile units 10, 30 to use in the height determination calculations, as discussed below. In other embodiments, the memory 36 of the mobile unit 30 may store the calibration data, which may be used in making height determinations based on information received from the fixed unit 10. In some embodiments, the memory 36 of the mobile unit 30 may store the various height measurements and determinations of subjects, though in at least one preferred embodiment these height measurements and determinations are not retained in memory for privacy or security reasons. In at least one embodiment, the mobile unit logic board 32 controls the operation of the entire system 100 as directed by input from a user 6, including when the fixed unit 10 performs the calibration, marking and measuring steps.

The mobile unit 30 may also include a display 39 that presents information to the user 6 of the system 100. The display 39 may be a screen, such as a digital screen, having any level of resolution capable of presenting information to the user 6 in a legible format which may include text, images and graphical representations of the data and/or status of the system 100, such as but not limited to whether the system 100 is in calibration, measure or marking mode; the status of the fixed unit 10, such as whether it is on, off or in standby mode awaiting instructions; the status of the mobile unit 30, such as whether it is on, off or in standby mode awaiting instructions or input from the user 6; the calibration value for the room, which is also referred to interchangeably herein as the known preselected distance; the height of the subject as determined by the system 100; the status of transmitting the subject's determined height to a hub 50 or EMR 60; the status or operational mode of the hub 50, such as on, off, receiving information or in standby mode. The display 39 may also include a speaker(s) providing audio information to a user 6, such as but not limited to a sound, beep, audio of a spoken language and the like.

The mobile unit 30 also includes input/output capabilities, such as at least one input sensor 35, to receive input from a user 6. For instance, in at least one embodiment, the input sensor(s) 35 is a sensor or switch in the display 39, such as a touchscreen display responsive to haptic input, that detects or registers the contact of a user 6 with the display 39 screen and transmits the corresponding input data and/or indicated instructions to the mobile unit logic board 32 for processing and further directing of output components, such as the mobile unit transceiver 38. In at least one other embodiment, the input sensor(s) 35 may be a button or plurality of buttons on the mobile unit 30 that may be selectively actuated by the user 6 to instruct the performance of calibration, marking and/or measuring by the system 100. The button(s) as input sensor 35 may be pressed or pressed and held for a predetermined length of time, to activate. For instance, in at least one embodiment as shown in FIG. 2, a first input sensor 35 may be pressed and held for 3 seconds, 5 seconds, or 10 seconds to indicate input instructions for calibrating. A second input sensor 35′ may be pressed to indicate input instructions for marking. The first sensor 35 may again be activated, this time by simply pressing to indicate input instructions for measuring. In other embodiments, there may be dedicated input sensors 35, 35′, 35″ for each mode of calibration, marking and measuring. In still other embodiments, there is a single input sensor 35 that is activated differently to indicate different input instructions for the various modes. These button(s) as input sensors 35, 35′, 35″ may be mechanical devices, such as switches or keys, or may be digital or electronic switches such as defined areas of a touchscreen display 39. Accordingly, at least a portion of the input sensor(s) 35, 35′, 35″ is accessible at the exterior of the mobile unit 30 for selective actuation by the user 6. As an I/O device, the input sensor(s) 35, 35′, 35″ is also capable of generating a signal to the mobile unit logic board 32 when activated according to the identity of the sensor and/or mode of activation.

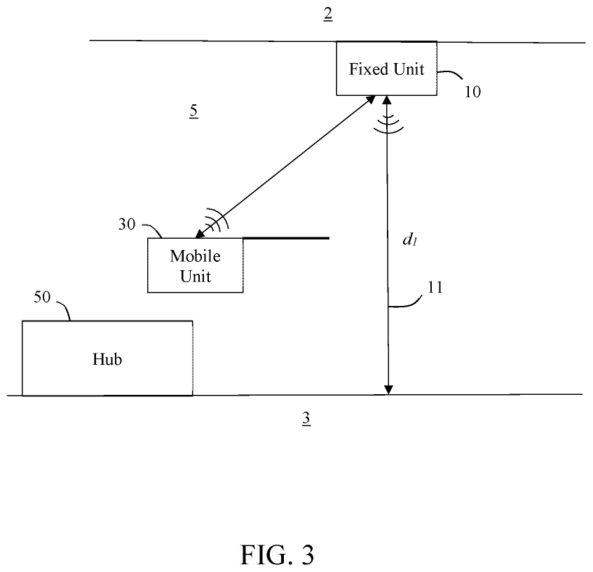

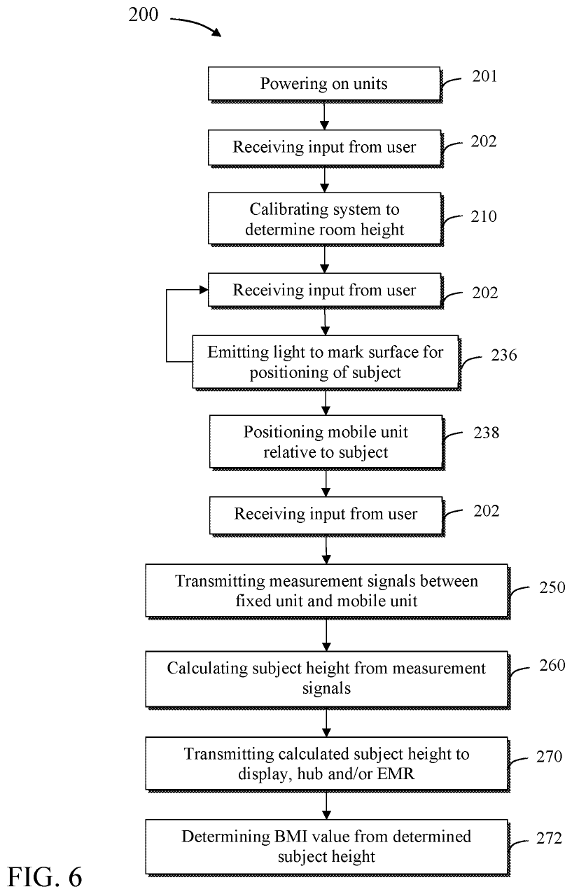

The present invention is also directed to methods of determining the height of a subject, as at 200, using the system 100 described above. This is shown schematically in FIGS. 6-8 and illustrated in FIGS. 3-5. For instance, and with reference to FIGS. 3 and 6, the method 200 includes first powering on the fixed unit 10 and mobile unit 30, as at 201. This may include turning the devices on from an off position or waking the devices from a standby or sleep mode. In at least one embodiment, powering on the devices includes manually turning on the mobile unit 30 and inputting instructions in the mobile unit 30 to activate or turn on the fixed unit 10. These instructions may be sent to the fixed unit 10 through the communications modules 38, 19 of the mobile unit 30 and fixed unit 10, respectively, such as wirelessly. In other embodiments, such as when the devices are in standby mode, the fixed unit 10 may receive a signal to turn on automatically when the mobile unit 30 enters the space 5 or is within transmission range of the fixed unit 10. Such signal may be sent automatically by the mobile unit 30. In still other embodiments, the fixed unit 10 may turn on or come out of standby when a subject 8 is detected in the vicinity, and the mobile unit 30 may turn on or come out of standby when picked up or touched by the user 6. In other embodiments, the hub 50 may send instructions to the mobile unit 30 and/or the fixed unit 10 to turn on when the hub 50 is turned on, brought out of standby or brought within transmitting range of the devices, such as when the hub 50 is mobile and wheeled into the room 5 or when the hub 50 is a workstation or examination table within the space 5 that is turned on or brought out of standby.

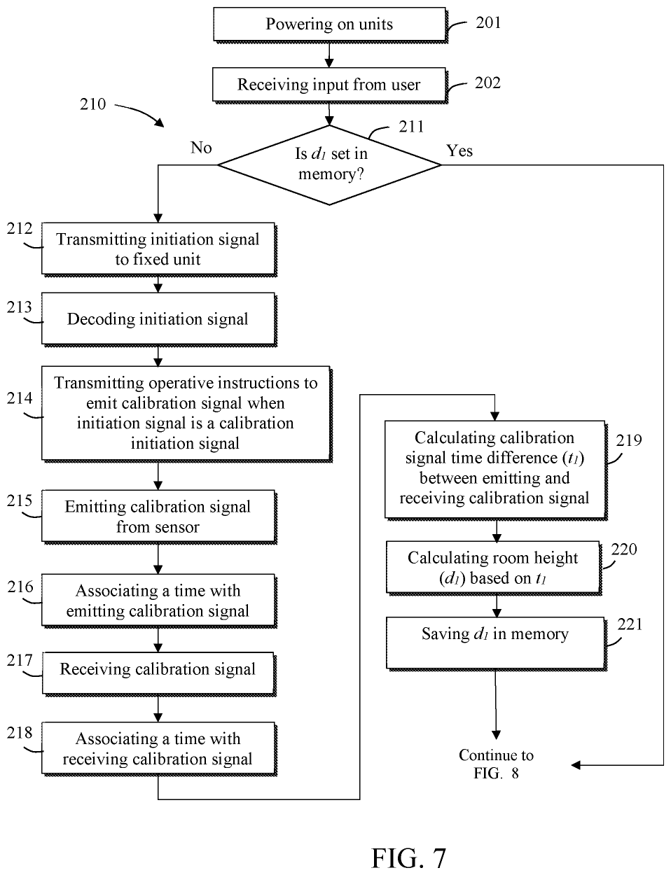

Once the fixed and mobile units 10, 30 are powered on, the method 200 continues with receiving input from the user, as at 202. This input from the user may be any interaction with the mobile unit 30, indicating the system 100 is both on and now active. Upon receiving initial input from the user, the system checks to see if calibration data is set in memory, as at 211 in FIG. 7. The calibration data is the distance (d₁) between the fixed unit 10 and the support substrate 3 and is also referred to herein as the known preselected distance or the room height. It may be determined by the method 200 described herein, or it may be already known, such as from manual measurement, and stored or coded into memory. In all instances, it is referred to in the Figures and calculations as d₁. The memory checked may be the memory 16 of the fixed unit 10, the memory 36 of the mobile unit 10, or both, or even the memory of the hub 50. If it is stored in multiple locations, additional rules may also be stored for which location is prioritized in the event of inconsistencies. If calibration data is stored in memory, then the method 200 continues as described below. If not, such as if this is the first time the system 100 has been used, if it has not been used for a threshold amount of time, if the memory has been cleared of calibration data, or if recalibration is desired for any reason, the user 6 may interact with the input sensor 35 on the mobile unit 30 to provide instructions to calibrate the system 100. This begins the step of calibrating the system 210. In some embodiments, the display 39 may present information to the user 6 upon powering on that there is no calibration data stored in the memory 16, 36 and that calibration is required before measurements can begin. In other embodiments, the display 39 may notify the user of the lack of calibration data when a measurement is attempted and there is no calibration data stored. These are but a few non-limiting examples.

To calibrate the system, as at 210, the user may input instructions at the mobile unit 30, such as by pressing a button or selecting an area of the display 39. In at least one embodiment, the mobile unit 30 includes a dedicated calibration button or area of the display 39 as an input sensor 35 that may be selectively activated to begin the calibration process. In other embodiments, there may be a single button or area of the display 39 as a single input sensor 35 for all user input that may be activated or held for a predetermined length of time, such as but not limited to 3 seconds, 5 seconds, 10 seconds or other predefined length of time to indicate calibration instructions. This user input is translated into an initiation signal which is transmitted from the mobile unit to the fixed unit, as at 212 in FIG. 7. The initiation signal may be electronic or digital and is transmitted to the fixed unit through the mobile unit transceiver 38 of the mobile unit 30.

The initiation signal is in turn received by the fixed unit 10, such as by the overhead communication module 19, and relayed to the fixed unit logic board 12 and processor 14. There, the method continues with decoding the initiation signal, as at 213. The fixed unit processor 14 compares the initiation signal received from the mobile unit 30 to a plurality of predefined signal profiles stored in memory 16. Each of a calibration initiation signal, mark initiation signal and measurement initiation signal have a unique signal profile stored in memory. Each signal initiation profile consists of a first packet of information indicating how many bits of data are in the defined signal, and a second packet of information providing the substantive data. The first packet of information is used to instruct the processor 14 on how much of the subsequent signal should be captured and interpreted. The second packet provides the relevant information being transmitted in the signal. When any initiation signal is received at the processor 14, the processor 14 compares at least the second packet of information in the received initiation signal to the stored initiation signal profiles and identifies the received initiation signal according to which of the stored initiation signal profiles it matches. Therefore, when the received initiation signal matches the stored calibration initiation signal profile, the processor 14 decodes the received initiation signal as a calibration initiation signal and provides instructions to proceed with the calibration step 210.

Specifically, when the processor 14 decodes the initiation signal as a calibration initiation signal, it transmits operative instructions to the sensor 17 to emit a calibration signal, as at 214. These operative instructions cause the sensor 17 to activate and emit a calibration signal, as at 215, according to preconfigured settings or as contained within the operative instructions. Types and characteristics of the calibration signal 11 emitted are described above in connection with sensor 17. The method 200 also includes associating a time with emitting the calibration signal, as at 216. This may be accomplished by creating a timestamp when the calibration signal 11 is emitted from the sensor 17. In other embodiments, a timer may be started when the calibration signal 11 is emitted. Upon being emitted from the sensor 17, the calibration signal 11 is directed at the support substrate 3, preferably beneath the fixed unit 10 and in at least one embodiment reflects off the support substrate 3 and back to the sensor 17 in the fixed unit 10, as depicted in FIG. 3. Therefore, the method 200 continues with receiving the reflected calibration signal by the overhead sensor, as at 217. The sensor 17 is capable of both sending and receiving the calibration signal 11. When the reflected calibration signal 11 is received, a time is associated with receiving the calibration signal, as at 218. As before, this may be by creating a timestamp upon receipt of the signal or by stopping the timer function. The sensor then sends a signal to the fixed unit logic board 12 and processor 14 of the times associated with when the calibration signal 11 was emitted and when the reflected wave was received.

In at least one embodiment, calibrating the system, as at 210, continues by calculating the calibration signal time difference (t₁) between emitting and receiving the calibration signal, as at 219. In some embodiments, such as when timestamps of events are transmitted, this calculation may occur by subtracting the timestamp of emitting the signal from the timestamp of receipt of the reflected signal and defining the result as t₁. In other embodiments, such as when timer information is transmitted, this calculation may simply be defining the timer information as t₁.

Calibrating the system, as at 210, then continues with calculating the room height d₁ based on t₁, as at 220. To do this, the processor 14 performs the following calculation:

$${d}_{1} = ( \frac{{t}_{1}}{2} ) c + x \qquad ( 1 )$$

where x is zero and c is either the speed constant of the wave when the calibration signal is laser light, or c is equal to the following equation when the calibration signal is ultrasonic:

c=331.4+(0.606*T)+(0.0124*H) (2)

where T is the temperature of the space 5 in degrees Celsius and H is the humidity of the space 5 in grams per cubic meter. Accordingly, LIDAR may be used in at least one embodiment to calibrate the system 100 such as when a laser is used for the calibration signal. The values for both c and x are stored in memory, such as fixed unit memory 16. The speed constant c will depend on the type and frequency of the wave emitted for calibration. For example, the speed constant c will be the speed of light when the sensor 17 is a laser emitting light, whereas the speed constant c will be the speed of sound as adjusted for temperature and humidity when the sensor 17 is ultrasonic emitting ultrasonic frequencies, as shown in Equation 2. The distance d₁ between the fixed unit 10 and the support substrate 3 may be up to 13 ft if the sensor 17 is ultrasonic, or up to 100 ft if the sensor 17 is a laser, though other distances are also contemplated depending on the power capacity and wave emitting features of the sensor 17 used.

Once the calibration height d₁ is determined, calibration concludes with saving d₁ in memory, as at 221. This may be saved in the memory 16 of the fixed unit 10 or may be transmitted to the mobile unit 30 and stored in the memory 36 there or to the hub 50 and stored in local memory there. In some embodiments, the calibration data d₁ is stored in multiple memories for increased flexibility in use of the system 100.

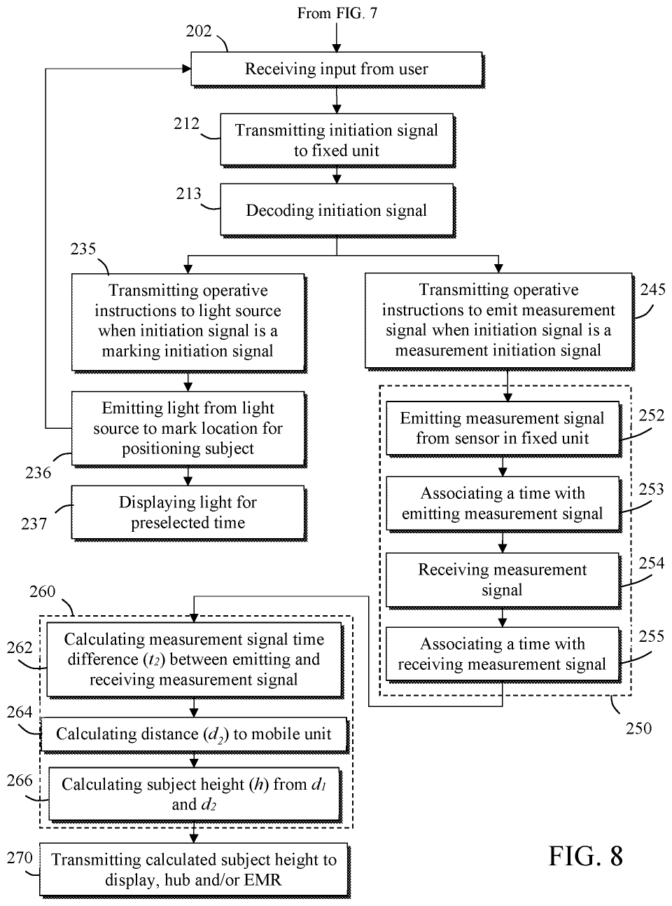

Once the system is calibrated, the method 200 continues with marking the location for the subject to stand, which begins with receiving input from the user, as at 202. This is shown schematically in FIGS. 6 and 8 and illustrated in FIG. 4. To accomplish this, the user actuates an input sensor 35 on the mobile unit 30, which may be a dedicated marking input button or area of the display 39 in some embodiments, or in other embodiments may be the sole input sensor 35 held for a predetermined length of time, such as but not limited to 1 second, 2 seconds, 3 seconds, or 5 seconds. In some embodiments, receiving marking input may occur by the user actuating the input sensor 35 in a certain pattern, such as but not limited to a predetermined number of times within a specified time period, such as 3 times within 5 seconds or 5 times within 10 seconds as a few non-limiting examples.

Upon receiving the marking input from the user, the method 200 continues with transmitting an initiation signal indicative of the user input to the fixed unit, as at 212 in FIG. 8. Here, the mobile unit 30 sends the marking initiation signal to the fixed unit 10 through their respective transceivers 38, 19. As noted previously, this may be transmitted wirelessly, such as but not limited to over a WiFi network, though it may be a wired communication in some embodiments. Once received in the fixed unit transceiver 19, the initiation signal is transmitted to the fixed unit logic board 12 and/or processor 14 where it is decoded, as at 213. As explained above, this decoding step may include parsing the packets of information in the initiation signal, such as for the size of incoming data and the corresponding data itself. Once decoded, the data communicated in the marking initiation signal is compared to a stored signals in memory 16. When the marking initiation signal received matches the stored marking initiation signal, it is identified as a marking initiation signal.

The method 200 then continues with transmitting operative instructions to the light source when the initiation signal is a marking initiation signal, as a 235. These operative instructions may include instructions to activate the light source 18 for a predetermined or communicated length of time, in a particular pattern and/or at a particular frequency, as described above in discussing the light source 18. In at least one embodiment, the operative instructions provided by the fixed unit logic board 12 to the light source 18 include instructions to activate the light source 18 for 2-3 seconds, 3-10 seconds, or 5 seconds then deactivate. In other embodiments, the light source 18 need not deactivate after a predetermined period of time, but rather may remain activated during the subsequent measuring step since it may not affect the measurement signals. During this activation time, the light source 18 emits light to mark the location for positioning the subject, as at 236 in FIGS. 6 and 8. The light 21 emitted from the light source 18 is projected to the support substrate 3 of the space 5 according to the orientation of the mounted fixed unit 10, creating a mark 20 on the support substrate 3, as shown in FIG. 4. In this manner, the method 200 includes displaying the mark for a preselected period of time, as at 237. In at least one embodiment, the light 21 is emitted from the light source 18 substantially perpendicular to the support substrate 3 beneath the fixed unit 10, as shown in FIG. 4. In other embodiments, the light 21 may be emitted from the light source 18 at non-perpendicular angles, such as within a cone expanding from the light source 18 to the support substrate 3, having a wider diameter at the support substrate 3 than at the light source 18, such as depicted in dotted lines in FIG. 4. Possible angles include but are not limited to up to 45° from normal to the support substrate 3.

Regardless of how light is emitted, the mark 20 denotes where the subject should stand for the system to subsequently determine their height. In at least one embodiment, the light is visible light and is colored so it is noticeable to the subject so they know where to stand and to the user so they can direct and assist the subject in positioning themselves over the mark 20. In some embodiments, the mark 20 may be displayed on a portion of the hub 50 when the subject 8 is to stand on the hub 50 of a component thereof for measurement, which in some embodiments may also have an integrated scale for obtaining weight measurements which may or may not occur simultaneously with the height determination. Regardless of where displayed, in some embodiments, the mark 20 may be larger than the size of the subject 8, such that the subject 8 stands within the perimeter or boundary of the mark 20. For example, the mark 20 may be in the range of 3-5 feet in diameter. In other embodiments, the mark 20 may be smaller than the size of the subject 8 such that the subject 8 stands over the mark 20, at least partially obscuring part of the mark 20 in doing so. Examples of such marks 20 may include but are not limited to up to 2 feet in diameter. In some embodiments, the light 21 producing the mark 20 may be emitted in a constant stream, producing a constant mark 20 for the duration of emission, such as but not limited to for 1 second, 1 minute, or the duration of use of the system 100, though other time intervals are also contemplated. In other embodiments, the light may be emitted in pulses, such as but not limited to 10-100 milliseconds or up to 1 second each in duration, repeating for the length of time the light is emitted. In certain embodiments, the light source 18 may include a filter or may be configured with a particular design stored in memory to create a pattern in the resulting mark 20 when it appears on the support substrate 3. This design of the mark 20 may be any shape, pattern, icon, image, text, number(s), or logo. In some embodiments, the mark 20 may be a common shape, such as but not limited to a diamond, triangle or square. In other embodiments, the mark 20 may be the logo of the user's employer, such as the medical practice or hospital. In other embodiments, the mark 20 may be a common icon such as but not limited to a smiley face. In still other embodiments, the mark 20 may be text or alphanumeric text, such as but not limited to the words “stand here” with a numerical countdown until the mark 20 disappears. These are a few non-limiting examples provided for illustration only. Preferably, the mark 20 is temporary, persisting only for a predetermined period of time according to the operative instructions or preset configuration provides, such as but not limited to in the range of 10 milliseconds-1 minute. This saves power, bulb or diode life, and ensures the measurement signals are not impeded by the mark 20 light. If the mark 20 disappears before the subject is properly positioned, the user may again activate the input sensor 35 to provide marking input to the mobile unit 30. This will repeat the above steps and generate another mark 20 on the support substrate 3. This process may be repeated as many times as necessary to achieve the proper positioning of the subject over the mark 20.

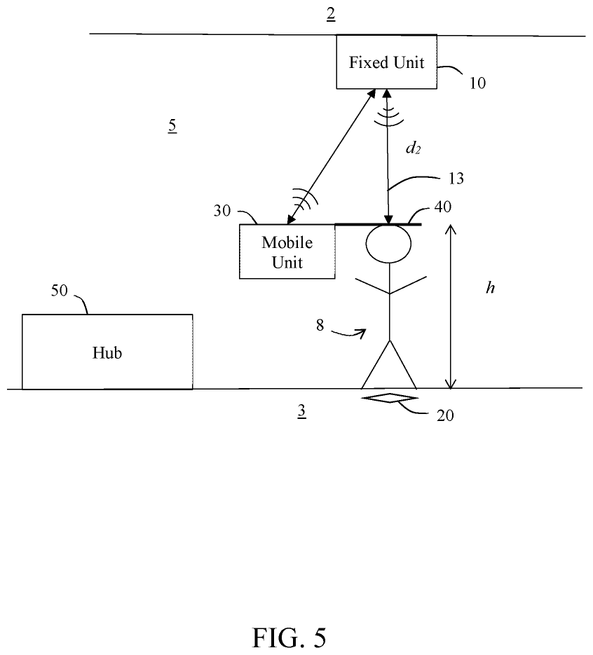

When the subject is positioned in the location of the mark 20, the method 200 continues with positioning the mobile unit relative to the subject, as at 238 in FIGS. 6 and 8. In this step, the user places at least a portion of the mobile unit 30 in proximity to a maximal point the subject, such as the head of the subject. In at least one embodiment, the measurement surface 40 of the mobile unit 30 is placed in a resting position on top of the head of the subject 8, as illustrated in FIG. 5. In some embodiments where the measurement surface 40 extends from the mobile unit 30, such as a paddle or arm which may be releasably attached to the mobile unit 30, this portion of the mobile unit 30 is placed on top of the subject's head. In other embodiment, such as when the display 39 is the measurement surface 40, the mobile unit 30 may be placed on the subject's head with the measurement surface 40 facing the fixed unit 10. Care should preferably be taken not to apply pressure when so placing the mobile unit 30 so as not to interfere with the subsequent measurement. The measurement surface 40 should be placed on, adjacent or in proximity to the part of the head desired to be used for height measurement purposes. For instance, the measurement surface 40 should preferably be placed on the highest part of the head in at least one embodiment, such as the crown, rather than the forehead, occipital bone or back of the head which may be lower and result in an inaccurate height measurement. Placing the measurement surface 40 directly on the head may have the benefit of pressing the subject's hair down so subsequent measurement is not affected by hair or hair style and is an accurate representation of the height of the subject 8. In some embodiments, such as when the hair or hair style does not permit direct contact with the head, the measurement surface 40 may be positioned adjacent to the head of the subject 8, rather than on top of it, so long as the measurement surface 40 is even with the desired point of the subject's head for measurement purposes.

Once the measurement surface is positioned appropriately, the method 200 continues with again receiving input from the user, as at 202 in FIGS. 6 and 8. This is accomplished by the user actuating the input sensor 35 as described above, this time indicating measurement. In at least one embodiment, there may be a dedicated input sensor 35 for measuring and the user actuated this measuring input sensor, such as by pressing it. In other embodiments where there is only a single input sensor 35 or where there are multiple input sensors actuated differently to produce different signals, the input sensor 35 may be actuated a single time, such as by pressing and immediately releasing, rather than holding down. Either of these will cause the mobile unit 30 to generate an initiation signal which is then transmitted to the fixed unit, as at 212 in FIG. 8. As with the other signals, this transmission is accomplished between the mobile unit transceiver 38 and fixed unit transceiver 19, which may be wireless or wired. In at least one embodiment, the transmission is wireless such as but not limited to over a WiFi network.

The method 200 continues with decoding the initiation signal, as at 213 in FIG. 8. As with the other initiation signals discussed above, the measurement initiation signal includes a unique set of packets of information, such as one indicating the size of incoming data and a second for the corresponding data itself. The initiation signal is decoded as described previously. The data communicated in the measurement initiation signal is compared to a stored measurement initiation signal in memory 16. When the measurement initiation signal received matches the stored measurement initiation signal, it is identified as a measurement initiation signal.

The method 200 then continues with transmitting operative instructions to the sensor to emit a measurement signal when the initiation signal is a measurement initiation signal, as at 245. These operative instructions may include instructions to activate the wave emitter and generate a wave, according to parameters included in the operative instructions, as configured in the sensor 17, or as set in the memory 16 of the fixed unit 10 or memory onboard the sensor 17. The various parameters are discussed above in connection with the sensor 17. For instance, in at least one embodiment, the operative instructions direct the sensor 17 to generate and emit a measurement signal 13 which may be the same as those described above for the calibration signal 11.

Once activated with operative instructions, the method 200 then continues with transmitting measurement signals between the fixed unit and mobile unit, as at 250 in FIGS. 6 and 8. This step includes emitting a measurement signal from the sensor in the fixed unit, as at 252 in FIG. 8. This measurement signal 13 is as described above and may be the same or different frequency as the calibration signal 11. The measurement signal 13 is emitted from the sensor 17 of the fixed unit 10 according to its mounted position on the surface 2. For instance, the sensor 17 is directed inwardly toward the space 5 such that the measurement signal 13 is emitted downward from the surface 2 toward the support substrate 3 in at least one embodiment, as shown in FIG. 5. In a preferred embodiment, the emitted measurement signal 13 is directed to the same location where the light 21 was previously emitted to produce the mark 20 on the support substrate 3, and hence where the subject is positioned. Accordingly, the emitted measurement signal 13 is preferably directed at the top of the subject's head. In certain embodiments, the light source 18 and sensor 17 may be the same. In at least one embodiment, the measurement signal 13 is transmitted from the sensor 17 substantially perpendicular to the support substrate 3 of the space and/or fixed unit 10, as shown in FIG. 5. This will reduce or eliminate the need to correct for an angle in the height calculations and is an improvement over other known height measuring systems, particularly in embodiments where the measurement signal 13 is reflected back to the sensor 17. However, in some embodiments the measurement signal 13 may be emitted at a slight angle relative to normal, such that the measurement signal 13 need not be exactly perpendicular to the support substrate 3 and/or fixed unit 10. Tolerances of up to 15 degrees from the center axis may still be appropriately detected such as by reflection and being received by the sensor 17. Because the sensor 17 is receiving the reflected wave, there is no need to correct for any angle. This is the benefit of emitting the measurement signal 13 from a fixed unit 10. In some embodiments, the sensor 17 also associates a time with emitting the measurement signal, as at 253. This may include logging the time, creating a timestamp, or sending a signal to the fixed unit logic board 12 or processor 14 to create such a log or timestamp when it emits the measurement signal 13. In other embodiments, the sensor 17 begins a timer or sends a signal to the fixed unit logic board 12 or processor 14 to begin a timer when emitting the measurement signal 13.

Because the emitted measurement signal 13 is directed at the subject's head, where the measurement surface 40 of the mobile unit 30 is positioned, the measurement signal 13 encounters and intercepts the measurement surface 40 when it reaches the mobile unit 30. In at least one embodiment, the measurement signal 13 bounces off the measurement surface 40 and is returned to the sensor 17 of the fixed unit 10 for detection, as shown in FIG. 5. This reflection by the measurement surface 40 of the mobile unit 30 may be active or passive. It may reflect the entire measurement signal 13 or portions thereof back to the sensor 17. For instance, if the measurement signal 13 is emitted from the sensor 17 at a slight angle, such as within the noted tolerance above, a portion of the measurement signal 13 may be reflected and returned back to the sensor 17 and another portion of the measurement signal 13 may be deflected at an angle. As long as at least a portion of the measurement signal 13 is returned to and detected by the sensor 17, that is sufficient for the purposes of this invention. As noted previously, this measurement surface 40 may be a paddle, arm or other extension from the mobile unit 30, or it may be the display 39 or other surface of the mobile unit 30 itself.

In other embodiments, however, the measurement surface 40 of the mobile unit 30 may not reflect back the measurement wave 13 upon intercepting it but may absorb the signal instead. For instance, the measurement surface 40 may include a detector 37 that detects the incident measurement signal 13. This detector 37 may operate actively or passively.

Accordingly, the method 200 includes receiving the measurement signal, as at 254 in FIG. 8. As noted previously, the sensor 17 is capable of both emitting and receiving waves, including the measurement signal 13. Therefore, in at least one embodiment the measurement signal 13 is reflected by the measurement surface 40 of the mobile unit 30 and travels at an angle sufficient to intercept the sensor 17 of the fixed unit 10. The sensor 17 therefor receives the reflected measurement signal 13. In other embodiments where the measurement surface 40 does not reflect the measurement signal 13, it is instead received by the mobile unit 30, specifically the measurement surface 40 and/or detector 37 therein.

The method 200 also includes associating a time with receiving the measurement signal, as at 255. In embodiments where a timestamp or log was created on emission, the sensor 17 or mobile unit 30 logs the time, creates a timestamp, or sends a signal to the fixed unit logic board 12 or processor 14 to create such a log or timestamp when the measurement signal 13 is received, depending on which unit is to receive the measurement signal 13 for detection. In embodiments involving a timer, the timer is stopped or a signal is sent to the fixed unit logic board 12 or processor 14 to stop the timer upon receiving the measurement signal 13.

The method 200 continues with calculating the subject's height from the measurement signals, as at 260 in FIGS. 6 and 8. This includes first calculating the measurement signal time difference (t₂) between emitting and receiving the measurement signal, as at 262 in FIG. 8. In at least one embodiment, the time stamps for the emitted and received measurement signal 13 are used in this calculation, subtracting the emitted wave timestamp from the received wave timestamp to provide the time difference t₂. In other embodiments, the time difference t₂ is simply calculated as the equivalent of the time logged in the timer. In at least one embodiment, the processor 14 or fixed unit logic board 12 of the fixed unit 10 may perform the calculation of time difference t₂. In other embodiments, the sensor 17 itself may perform this calculation if it has sufficient processing capabilities or when it includes a timer that is used in determining the times of the measurement signal. In some embodiments, however, time stamps or timer value for the measurement signal 13 may be transmitted to and/or determined by the mobile unit 30 for the mobile unit logic board 32 or processor 34 to perform the calculation of time difference t₂. Once calculated, measurement signal time difference t₂ may be stored in memory 16, 36 or onboard the sensor 17 for future use. Accordingly, the time difference t₂ may be transmitted to the other device 10, 30 from where it was determined for storage, either permanently or temporarily. In still other embodiments, the times for emitting and receiving the measurement signal may be sent to the hub 50 for calculation of the measurement signal time difference t₂ where it may be stored in local memory.

Once the measurement signal time difference t₂ is calculated or determined, the method 200 then includes calculating the distance (d₂) to the mobile unit, as at 264. This distance d₂ is the distance between the sensor 17 emitting the measurement surface and the measurement surface 40 of the mobile unit 30 which intercepted and, in some embodiments, returned the reflected measurement signal 13. It is therefore also the distance the measurement signal 13 traveled. Because the measurement surface 40 of the mobile unit 30 is also placed on, adjacent to or in close proximity to the head of the subject 8, the distance d₂ also represents the distance between the sensor 17 and the subject's head. This calculation is performed by the following equation:

$${d}_{2} = ( \frac{{t}_{2}}{2} ) c + x \qquad ( 3 )$$

When the measurement signal is laser light emitted from the sensor 17, x is zero and c is the speed of light. When the measurement signal is ultrasonic waves emitted from the sensor 17, x is either the height dimension of the measurement surface 40 of the mobile unit 30 and c is equal to the following equation:

c=331.4+(0.606*T)+(0.0124*H) (4)

where T is the temperature of the space 5 in degrees Celsius and H is the humidity of the space 5 in grams per cubic meter. In embodiments where the sensor 17 is ultrasonic, the height x may be up to 1.5 inches, such as but not limited to in the range of 0.25-1.5 inches, and in some embodiments 0.25-0.375 inch. Could also be thicker, such as up to 1.5 inches, for instance when a smartphone or tablet is used as the mobile unit and measurement surface. Accordingly, LIDAR may be used in at least one embodiment to calculate the distance d₂ to the subject, such as when a laser is used to generate the measurement signal. As with the measurement signal time difference t₂, either the fixed unit logic board 12, processor 14 or mobile unit logic board 32 or processor 34 may perform the calculation of distance dz. In some embodiments, it may be beneficial or desired for the fixed unit 10 to perform all the calculations, such as to keep the processing power focused in one location, allowing the mobile unit 30 to be smaller, lighter, faster or more portable. In other embodiments, it may be beneficial or desired for the mobile unit 30 to perform the calculations, such as to have the ability to move between rooms with different fixed units 10 and retain the processing power and stored information for increased mobility. In still other embodiments, it may be preferred for the hub 50 to perform the calculations. Regardless of which device 10, 30, 50 performs the calculations, the necessary values and measurements for such calculations are captured and/or transmitted to the relevant device 10, 30, 50 through the transceivers 19, 38 of the fixed and mobile units 10, 30 for computation.

Finally, the method 200 includes calculating the height (h) of the subject from d₁ and d₂, as at 266. This height calculation is performed according to the following formula:

h=d₁−d₂ (5)

where d₁ is the known preselected distance or calibrated measurement of the height of the space and d₂ is the distance the measurement signal 13 traveled between the sensor 17 of the fixed unit 10 and the measurement surface 40 of the mobile unit 30. The difference between these two values is the height h of the subject 8. Because each of the distances d₁ and d₂ already account for the height of the measurement surface 40, the resulting subject height h is also accurate.

The method 200 also preferably includes transmitting the calculated subject height h to the display, hub and/or EMR, as at 270 in FIGS. 6 and 8. Regardless of which device 10, 30 performs the height calculations, the resulting value may be sent to another component within the system 100 for viewing by the subject 8, user 6, or health professional through the hub 50 or EMR 60 discussed previously. For instance, in some embodiments the calculated height h may be transmitted to the display 39 of the mobile unit 30 so the user 6 and/or subject 8 may readily see the value. The display 39 may present the height h to the viewer in a variety of formats, such as but not limited to digital, graphical, numerical, pictographic, audio and/or other format that is readily understood by the viewer, which may or may not include units. Any relevant units of measure for height may be used, such as but not limited to feet, inches, meters, and centimeters. The calculated height h may also be transmitted to the hub 50 located within the space 5, as shown in FIG. 5, such as when the hub 50 is defined as a central repository for health or medical information for the subject 8 and may in some embodiments have processing capabilities to determine the BMI of the subject 8 once the height h value is received. The calculated height h may also be transmitted to the subject's EMR 60 as described previously, to be added to their EMR 60. It may also include the date and time of the measurements and calculations for recording purposes.

In some embodiments, the method 200 may also include determining the body mass index (BMI) value of the subject from the determined height of the subject, as at 272. The BMI may be determined by the hub 50, user 6, or another source from the calculated height h and weight information collected, such as by the hub 50 or a scale in electronic communication with the components of the system 100. The BMI value may be determined as understood in the medical arts according to the relationship between height and weight. The BMI value as determined may also be transmitted to the EMR 60 for recording purposes and future use by medical or health practitioners and professionals. Accordingly, the calculated height h may be sent to any or all of the display 39, hub 50, and EMR 60 according to the system 100 configuration.

Since many modifications, variations and changes in detail can be made to the described preferred embodiments, it is intended that all matters in the foregoing description and shown in the accompanying drawings be interpreted as illustrative and not in a limiting sense. Thus, the scope of the invention should be determined by the appended claims and their legal equivalents. Now that the invention has been described,

What is claimed is:

1. A method of determining the height of a subject, said method comprising:
positioning said subject at a predetermined location on a support substrate relative to a fixed unit;
positioning a mobile unit in proximity to a maximal point of said subject at said predetermined location;
generating and emitting a measurement signal from said fixed unit directed at said subject at said predetermined location;
receiving said measurement signal;
determining a distance between said fixed unit and said mobile unit based on said measurement signal;
determining a height of said subject by subtracting said distance between said fixed unit and said mobile unit from a known preselected distance from said fixed unit to said support substrate under said subject; and
transmitting a signal indicative of said height of said subject.

2. The method as recited in claim 1, further comprising associating a time with emitting said measurement signal; associating a time with receiving said measurement signal; determining a measurement signal time difference between emitting and receiving said measurement signal; and determining a distance between said fixed unit and said mobile unit based on said measurement signal time difference.

3. The method as recited in claim 1, further comprising generating an initiation signal at one of said mobile unit and said fixed unit, said initiation signal directing generating and emitting one of: (a) said measurement signal, (b) a calibration signal, and (c) a marking signal.

4. The method as recited in claim 3, further comprising decoding said initiation signal to identify said initiation signal as one of: (a) a measurement initiation signal, (b) a calibration initiation signal, and (c) a marking initiation signal.

5. The method as recited in claim 4, further comprising generating and emitting a measurement signal from said fixed unit in response to said measurement initiation signal; generating and emitting a calibration signal from said fixed unit in response to said calibration initiation signal; and generating and emitting a marking signal from said fixed unit in response to said marking initiation signal.

6. The method as recited in claim 3, wherein said initiation signal is caused by user input at said mobile unit.

7. The method as recited in claim 6, further comprising transmitting said initiation signal from said mobile unit to said fixed unit.

8. The method as recited in claim 1, further comprising generating and emitting a calibration signal from said fixed unit directed at said support substrate; receiving said calibration signal at said fixed unit as returned by said support substrate; and determining a distance between said fixed unit and said support substrate based on said calibration signal.

9. The method as recited in claim 8, further comprising storing said distance between said fixed unit and said support substrate as said known preselected distance in memory.

10. The method as recited in claim 8, further comprising associating a time with emitting said calibration signal; associating a time with receiving said calibration signal; determining a calibration signal time difference between emitting and receiving said calibration signal; and determining said distance between said fixed unit and said support substrate based on said calibration signal time difference.

11. The method as recited in claim 1, further comprising checking memory for said known preselected distance; and performing the following steps when said known preselected distance is not stored in memory:
(i) generating and emitting a calibration signal from said fixed unit directed at said support substrate;
(ii) receiving said calibration signal at said fixed unit as returned by said support substrate; and
(iii) determining said distance between said fixed unit and said support substrate based on said calibration signal; and
(iv) storing said distance between said fixed unit and said support substrate in said memory as said preselected distance.

12. The method as recited in claim 1, further comprising generating and emitting a marking signal from said fixed unit directed at said predetermined location of said support substrate; and
displaying a mark on said predetermined location of said support substrate from said marking signal.

13. The method as recited in claim 12, further comprising displaying said mark for a preselected period of time.

14. The method as recited in claim 13, wherein said preselected period of time is less than 10 seconds.

15. The method as recited in claim 13, wherein emitting said marking signal includes one of (i) constantly emitting said marking signal for said predetermined period of time, and (ii) pulsing said marking signal for said preselected period of time.

16. The method as recited in claim 15, wherein pulsing said marking signal occurs in increments in the range of 10 to 100 milliseconds for the duration of said preselected period of time.

17. The method as recited in claim 1, wherein determining said height of said subject is performed by one of said fixed unit, said mobile unit, and a hub in electronic communication with at least one of said fixed unit and said mobile unit.

18. The method as recited in claim 17, wherein determining a distance between said fixed unit and said mobile unit is performed by one of said fixed unit, said mobile unit, and said hub.

19. The method as recited in claim 1, further comprising transmitting said height of said subject as determined by said method to one of a hub, electronic medical record and a display of said mobile unit.

20. The method as recited in claim 1, further comprising determining a body mass index value for said subject from said height of said subject as determined by said method.

21. The method as recited in claim 20, wherein said body mass index value is determined by one of said hub, said mobile unit and said fixed unit.

22. A system for determining the height of a subject, comprising:
a fixed unit affixed to a surface of a space, said fixed unit including:
(i) a fixed unit transceiver capable of sending and receiving electronic signals; and
(ii) a sensor generating and emitting a measurement signal directed at said subject at a predetermined location when said sensor is activated, said sensor further capable of receiving said measurement signal when said measurement signal is reflected back to said sensor;
a mobile unit movably positionable within said space, said mobile unit including:
(iii) a mobile unit transceiver in electronic communication with said fixed unit transceiver, said mobile unit transceiver capable of sending and receiving electronic signals; and
(iv) a measurement surface selectively positionable in proximity to a maximal point of said subject and intercepting said measurement signal; and
a logic board having a processor and memory, said logic board associated with one of said fixed unit and said mobile unit, said logic board:
(v) determining a distance between said fixed unit and said mobile unit based on said measurement signal; and
(vi) determining a height of said subject based on said distance and a known preselected distance from said fixed unit to a support substrate.

23. The system as recited in claim 22, wherein said measurement signal is one of ultrasonic, sonic, visible light and UV light.

24. The system as recited in claim 22, wherein said sensor is a laser.

25. The system as recited in claim 22, wherein said mobile unit further comprises at least one input sensor at an exterior of said mobile unit, said at least one input sensor receiving operative input from a user, said mobile unit transceiver transmitting an initiation signal indicative of said operative input from said user to said fixed unit.

26. The system as recited in claim 25, wherein said initiation signal is one of a calibration initiation signal, a marking initiation signal, and a measurement initiation signal as directed by said operative input from said user.

27. The system as recited in claim 25, wherein said sensor generates and emits a calibration signal directed at said support substrate when said sensor is activated for calibration, wherein said calibration signal is one of ultrasonic, sonic, visible light and UV light.

28. The system as recited in claim 22, wherein said measurement surface of said mobile unit is one of (i) a surface of said mobile unit, and (ii) extends from said mobile unit.

29. The system as recited in claim 28, wherein said measurement surface is selectively attachable to and removable from said mobile unit.

30. The system as recited in claim 22, wherein said mobile unit further comprises a display including at least one of (i) a screen, (ii) said measurement surface, and (iii) at least one input sensor.

31. The system as recited in claim 22, wherein said fixed unit further comprising a light source generating and emitting a light directed toward a support substrate when said light source is activated, said system further comprising a mark displayed on said support substrate indicating a position for said subject for measurement, said mark being said light from said light source when it reaches said support substrate.

32. The system as recited in claim 22, further comprising a hub in electronic communication with at least one of said fixed unit and said mobile unit.

33. The system as recited in claim 32, wherein at least one of said fixed unit, said mobile unit and said hub including a processor capable of determining at least one of (i) said height of said subject, and (ii) a body mass index for said subject from said height of said subject.

34. The system as recited in claim 33, further comprising an electronic medical record of said subject, wherein at least one of said fixed unit, said mobile unit and said hub are in electronic communication with said electronic medical record and transmitting at least one of said height and said body mass index of said subject to said electronic medical record.
